(19) United States
(12) Patent Application Publication
(10) Pub. No.: US 2009/0181421 A1
(43) Pub. Date: Jul. 16, 2009

(54) DIAGNOSIS OF FETAL ABNORMALITIES USING NUCLEATED RED BLOOD CELLS

(76) Inventors: Ravi Kapur, Stoughton, MA (US); Diana Bianchi, Charlestown, MA (US); Tom Barber, Allston, MA (US); Mehmet Toner, Wellesley Hills, MA (US)

Correspondence Address: PROSKAUER ROSE LLP, Suite 400 South, 1001 Pennsylvania Avenue, NW, Washington, DC, 20004, US

(21) Appl. No.: 12/172,158

(22) Filed: Jul. 11, 2008

Related U.S. Application Data
(63) Continuation-in-part of application No. 11/228,462, filed on Sep. 15, 2005.
Application No. 11/228,462 is a continuation-in-part of application No. 11/324,041, filed on Dec. 29, 2005.
(60) Provisional application No. 60/703,833, filed on Jul. 29, 2005.

(51) Int. Cl. C12Q 1/06

(52) U.S. Cl. 435/39

(57) ABSTRACT

The present invention relates to methods for diagnosing a condition in a fetus by enriching and enumerating circulating red blood cells with the possible combination of results from maternal serum marker screens.

### CROSS-REFERENCE

This application is a continuation-in-part application of Ser. No. 11/228,462, filed Sep. 15, 2005, and claims the benefit of U.S. Provisional Application No. 60/949,227, filed Jul. 11, 2007, which are incorporated herein by reference in their entirety.

### BACKGROUND OF THE INVENTION

The presence of fetal cells in the peripheral blood of a pregnant mammal provides an opportunity to practice prenatal diagnostics without the risks associated with more invasive diagnostic procedures. Fetal cells are quite rare in comparison to other cells found in peripheral blood. Enrichment of fetal cells from peripheral blood facilitates the analysis of these cells and makes it easier to diagnose fetal abnormalities.

### SUMMARY OF THE INVENTION

In general, the present invention relates to systems, apparatus, and methods for determining the presence of a fetal or maternal abnormal condition by enumerating fetal nucleated red blood cells isolated from a sample from a pregnant woman. Implementation of the invention can include one or more of the following features.

In general, in one aspect, a method for determining the presence of a fetal abnormal condition is provided, including enumerating nucleated red blood cells in a blood sample from a pregnant woman and determining the presence of a fetal abnormal condition based on the number of nucleated red blood cells in the blood sample.

In general, in another aspect, a method for determining the presence of aneuploidy in a fetus is provided, including a) enumerating nucleated red blood cells in a sample from a pregnant woman and b) assigning a likelihood of said pregnant woman's fetus being aneuploid based on statistical averages of nucleated red blood cells from blood samples from pregnant women carrying euploid fetuses compared with statistical averages of nucleated red blood cells from blood samples from pregnant women carrying aneuploid fetuses.

In general, in yet another aspect, a method for determining the presence of a fetal abnormal condition is provided including a) enumerating nRBCs in a first blood sample from a pregnant woman (b) and either: (i) detecting the presence or level of one or more serum markers in the first or a second blood sample from the pregnant woman, (ii) measuring space in nuchal fold of her fetus; or (iii) or both (i) and (ii), and determining the presence of the fetal abnormal condition in the fetus from results from steps (a) and (b).

In one embodiment, the method can include the step of enriching nucleated red blood cells from enucleated red blood cells or white blood cells. In another embodiment, the enriching can be based on cell size and/or magnetic property. In another embodiment, the enriching can include using arrays of obstacles. In another embodiment, the enriching can include rendering nucleated red blood cells magnetic. In another embodiment, the enriching can include using arrays of obstacles and rendering nucleated red blood cells magnetic.

In another embodiment, the sample can be taken in the first trimester of pregnancy. In another embodiment, the said pregnant woman can be under the age of 35. In another embodiment, the nRBCs can be enriched in a flow-through microfluidic device. In another embodiment, the enumerating of nRBCs can be performed by flow cytometry, fluorescence imaging, or radioactive imaging. In another embodiment, the method can further include performing fluorescence in situ hybridization on said nucleated red blood cells with chromosome-specific probes. In another embodiment, when the number of nRBCs and/or aneuploid nRBCs exceeds a pre-determined value, the method can include determining the genetic characteristics of said pregnant woman's fetus. In another embodiment, the serum markers can be comprised of papA, free β HCG, unconjugated estriol (UE3), AFP, HCG, or inhibin.

In another embodiment, the aneuploidy can be trisomy 21. In another embodiment, the aneuploidy can be trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY, or triploidy. In another embodiment, the fetal abnormal condition can be Klinefelter Syndrome, dup(17)(p11.2p1.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Prader-Willi syndrome, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p36 deletion, or a combination thereof.

In another embodiment, the method can include determining the origin of the cells enumerated in step (b).

In another embodiment, the sample can be a peripheral blood sample. In another embodiment, the sample can be an amniotic sample.

In general, in yet another aspect, a method for determining a condition in a fetus of a subject is provided, including enriching one or more nucleated red blood cells from a first sample from said subject, performing a maternal serum marker screen on said first sample or a second sample from said subject, optionally, performing a Nuchal Translucency (NT) sonographic test on said first sample, said second sample, or a third sample from said subject, determining a condition of said fetus based on: (1) the number of nucleated red blood cells isolated from said first sample, (2) the results from said maternal serum marker screen; and (3) optionally, the results from said Nuchal Translucency test.

In one embodiment, the condition can be selected from the group consisting of trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY, XYYYY, Klinefelter Syndrome, dup(17)(p1.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p36 deletion, or a combination thereof.

In another embodiment, the first sample, second sample, or third sample can be a peripheral blood sample.

In another embodiment, the maternal serum marker screen can be AFP, MSAFP, Double Marker Screen, Double Screen, Triple Marker Screen, Triple Screen, Quad Screen, 1st Trimester Screen, 2nd Trimester Screen, Integrated Screen, Combined Screen, Contingency Screen, Repeated Measures Screen or Sequential Screen.

In another embodiment, the subject can be under the age of 35. In another embodiment, the sample can be taken in the first trimester of pregnancy.

In another embodiment, the enriching can be based on cell size and/or magnetic property. In another embodiment, the enriching can include using arrays of obstacles. In another embodiment, the enriching can include rendering nucleated red blood cells magnetic. In another embodiment, the enriching can include using arrays of obstacles and rendering nucleated red blood cells magnetic.

In general, in yet another aspect, a method for determining the presence of a maternal abnormal condition is provided including enumerating nucleated red blood cells in a blood sample from a pregnant woman and determining the presence of a maternal abnormal condition based on the number of nucleated red blood cells in the blood sample.

In one embodiment, the condition can be Pre-eclampsia.

### BRIEF DESCRIPTION OF THE DRAWINGS

FIGS. 1A-1D illustrate some of the operational principles of a size-based separation module.

FIGS. 2A-2C illustrate one embodiment of an affinity separation module.

FIG. 3 illustrates one embodiment of a magnetic separation module.

FIGS. 4A-4D illustrate schematics of a size-based separation module.

FIG. 5A illustrates a schematic representation of a high-gradient magnet, designed to generate 1.2 Tesla to about 3 Tesla/mm.

FIG. 5B illustrates a schematic representation of a capillary disposed adjacent to the magnet shown in FIG. 5A.

FIG. 5C is a graph of the field strength of the magnet as a function of the position of the capillary.

FIG. 6 is a summary of the results of the Phase I study performed at Site B.

FIG. 7 is a summary of the results of the Phase I study performed at Site C.

FIG. 8 lists the descriptive statistics and effect sizes for the combined nRBC enumeration and number of certitude trisomic events data from both sites.

FIG. 9 is a summary of the results of a larger clinical study.

FIG. 10A is a summary of the results of a simulated clinical study.

FIG. 10B is a summary of the sensitivity rates calculated from the simulated clinical study.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention provides systems, apparatus, and methods to diagnose conditions in a fetus based on the number of nucleated red blood cells collected from a sample from the mother. Furthermore, the present invention also provides methods to diagnose or prognosticate a condition in a fetus based on the number of nucleated red blood cells (nRBCs) collected from a sample from a mother using the aforementioned systems, apparatus, and methods. Further, serum marker screen data and/or nuchal translucency data taken from the same mother can be combined with nucleated red blood cell enumeration to diagnose or prognosticate a condition in a fetus.

The invention also relates to a method for identifying a characteristic associated with a condition in a subject comprising obtaining a plurality of control samples, obtaining a plurality of case samples, applying each of said samples to a device comprising a plurality of obstacles that deflect a first analyte (such as a nucleated red blood cell or a trophoblast) from said sample in a direction away from a second analyte (such as an enucleated red blood cell) of said blood sample wherein said first analyte and said second analyte have a different hydrodynamic diameter, analyzing said first analyte from said samples to determine a characteristic of said first analyte, and performing an association study based on said characteristic.

### I. Sample Collection/Preparation

Samples containing rare cells can be obtained from a mammal pregnant with a fetus in need of a diagnosis or prognosis. In one example, a sample can be obtained from mammal suspected of being pregnant, pregnant, or that has been pregnant to detect the presence of a fetus or detect a fetal condition (such as an abnormal fetal condition). The mammal of the present invention can be a human or a domesticated mammal such as a cow, pig, horse, rabbit, dogs, cat, or goat. Samples derived from a mammal or human can include, e.g., whole blood, amniotic fluid, or cervical swabs.

To obtain a blood sample, any technique known in the art can be used (such as withdrawal with a syringe a hypodermic needle connected to a Vacutainer tube, or other vacuum device. A blood sample can be optionally pre-treated or processed prior to enrichment (such as by the addition of sodium heparin).

Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent. Examples of other processing steps prior to enrichment include density centrifugation or leukocyte reduction filters.

When a blood sample is obtained, a preservative such an anti-coagulation reagent and/or a stabilizer can be added to the sample prior to enrichment. This allows for extended time for analysis/detection. Thus, a sample, such as a blood sample, can be enriched and/or analyzed under any of the methods and systems herein within 30 days, 1 week, 6 days, 5 days, 4 days, 3 days, 2 days, 1 day, 23 hrs, 22 hrs, 21 hrs, 20 hrs, 19 hrs, 18 hrs, 17 hrs, 16 hrs, 15 hrs, 14 hrs, 13 hrs, 12 hrs, 11 hrs, 10 hrs, 9 hrs, 8 hrs, 7 hrs, 6 hrs, 5 hrs, 4 hrs, 3 hrs, 2 hrs, 1 hrs, 45 min, 30 min, 20 min, or 15 min from the time the sample is obtained.

In some embodiments, a blood sample can be combined with a reagent that selectively lyses one or more cells or components in the blood sample. For example, fetal nucleated cells can be selectively lysed releasing their nuclei when a blood sample comprising fetal nucleated cells is combined with deionized water. Such selective lysis allows for the subsequent enrichment of fetal nuclei using, e.g., size or affinity based separation. In another example platelets and/or enucleated red blood cells are selectively lysed to generate a sample enriched in nucleated cells, such as fetal nucleated red blood cells (fnRBC's) or maternal nucleated blood cells (mnBC). fnRBC's can be subsequently separated from mnRBC's or maternal nucleated red blood cells (mnRBC) using, e.g., antigen-i affinity or differences in hemoglobin.

The amount of sample collected (e.g., blood sample), can vary depending upon mammal size, its gestation period, and the condition being screened. In some embodiments, up to 50, 40, 30, 20, 10, 9, 8, 7, 6, 5, 4, 3, 2, or 1 mL of a sample is obtained. In some embodiments, 1-50, 2-40, 3-30, or 4-20 mL of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

To detect fetal condition, a blood sample can be obtained from a pregnant mammal or human within 36, 24, 22, 20, 18, 16, 14, 12, 10, 8, 6 or 4 weeks of gestation or even after a pregnancy has terminated.

### II. Enrichment

A sample (e.g. blood sample) can be enriched for nucleated RBC's or fetal nucleated cells (e.g., trophoblasts) using any methods known in the art (e.g. Guetta, E M et al. Stem Cells Dev, 13(1):93-9 (2004)) or described herein.

In some embodiments, enrichment occurs by selective lysis as described above.

In one embodiment, nRBCs (such as mnRBCs or fnRBCs) are enriched using one or more size-based separation modules. Nucleated RBCs can be infrequent in number compared to other nucleated cells found in maternal peripheral blood. In another embodiment enrichment of fetal trophoblasts occurs using one or more size-based separation modules. Examples of size-based separation modules include filtration modules, sieves, matrixes, etc. Examples of size-based separation modules contemplated by the present invention include those disclosed in International Publication No. WO 2004/113877, which is herein incorporated by reference in its entirety. Other size based separation modules are disclosed in International Publication No. WO 2004/0144651, which is herein incorporated by reference in its entirety. Yet other size based separation modules are disclosed in United States Publication No. US 2006-0223178 A1, which is herein incorporated by reference in its entirety.

In some embodiments, a size-based separation module comprises one or more arrays of obstacles forming a network of gaps. The obstacles are configured to direct particles (e.g. cells) as they flow through the array/network of gaps into different directions or outlets based on the particle's hydrodynamic size. For example, as a blood sample flows through an array of obstacles, nucleated cells or cells having a hydrodynamic size larger than a predetermined size (e.g., 4, 5, 6, 7, 8, 9, or 10 microns) are directed to a first outlet located on the opposite side of the array of obstacles from the fluid flow inlet, while the enucleated cells or cells having a hydrodynamic size smaller than a predetermined size (e.g., 4, 5, 6, 7, 8, 9, or 10 microns) are directed to a second outlet also located on the opposite side of the array of obstacles from the fluid flow inlet.

An array can be configured to separate cells smaller or larger than a predetermined size by adjusting the size of the gaps, obstacles, and offset in the period between each successive row of obstacles. For example, in some embodiments, obstacles or gaps between obstacles can be up to 10, 20, 50, 70, 100, 120, 150, 170, or 200 microns in length or about 2, 4, 6, 8, 10, 20, 30 or 40 microns in length. In some embodiments, an array of obstacles for size-based separation includes more than 100, 500, 1,000, 5,000, 10,000, 50,000, 100,000, or 200,000 obstacles that are arranged into more than 10, 20, 50, 100, 200, 500, 1000 or 2000 rows. Obstacles in a first row of obstacles can be offset from a previous (upstream) row of obstacles by up to 50% the period of the previous row of obstacles. In some embodiments, obstacles in a first row of obstacles are offset from a previous row of obstacles by up to 45, 40, 35, 30, 25, 20, 15 or 10% the period of the previous row of obstacles. Furthermore, the distance between a first row of obstacles and a second row of obstacles can be up to 10, 20, 50, 70, 100, 120, 150, 170 or 200 microns. A particular offset can be continuous (repeating for multiple rows) or non-continuous. In some embodiments, a separation module includes multiple discrete arrays of obstacles fluidly coupled such that they are in series with one another. Each array of obstacles can have a continuous offset. Each subsequent (downstream) array of obstacles can have an offset that is different from the previous (upstream) offset. For example each subsequent array of obstacles can have a smaller offset that the previous array of obstacles. This allows for a refinement in the separation process as cells migrate through the array of obstacles. Thus, a plurality of arrays can be fluidly coupled in series or in parallel (e.g., more than 2, 4, 6, 8, 10, 20, 30, 40, 50). Fluidly coupling separation modules (e.g., arrays) in parallel allows for high-throughput analysis of the sample, such that at least 1, 2, 5, 10, 20, 50, 100, 200, or 500 mL per hour flows through the enrichment modules or at least 1, 5, 10, or 50 million cells per hour are sorted or flow through the device.

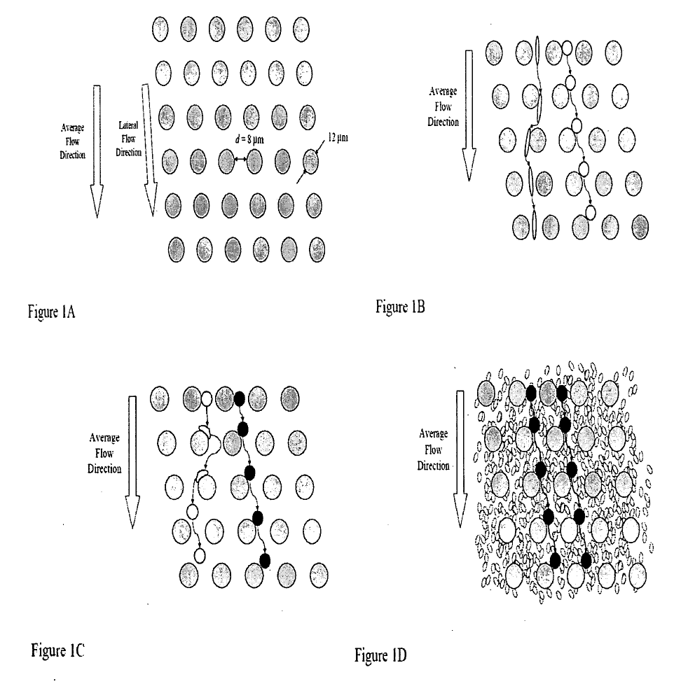

FIG. 1A illustrates an example of a size-based separation module. Obstacles (which can be of any shape) are coupled to a flat substrate to form an array of gaps. A transparent cover or lid can be used to cover the array. The obstacles form a two-dimensional array with each successive row shifted horizontally with respect to the previous row of obstacles, where the array of obstacles directs component having a hydrodynamic size smaller than a predetermined size in a first direction and component having a hydrodynamic size larger that a predetermined size in a second direction. For example enriching fetal cells or nRBC's from a mixed sample (e.g. maternal blood sample) the hydrodynamic size can be between 4-10 μm or between 6-8 μm. The flow of sample into the array of obstacles can be aligned at a small angle (flow angle) with respect to a line-of-sight of the array. Optionally, the array is coupled to an infusion pump to perfuse the sample through the obstacles. The flow conditions of the size-based separation module described herein are such that cells are sorted by the array with minimal damage. This allows for downstream analysis of intact cells and intact nuclei to be more efficient and reliable.

A size-based separation module comprising an array of obstacles can be configured to direct cells larger than a predetermined size to migrate along a line-of-sight within the array (e.g. towards a first outlet or bypass channel leading to a first outlet), while directing cells and analytes smaller than a predetermined size to migrate through the array of obstacles in a different direction than the larger cells (e.g. towards a second outlet). Such embodiments are illustrated in part in FIGS. 1B-1D. For example, nRBC's are directed to a first output while enucleated RBC's are directed to a second output.

While a variety of enrichment protocols can be utilized, gentle handling of the cells can reduce any mechanical damage to the cells or their DNA. This gentle handling can serve to preserve the small number of fetal cells or nucleated red blood cells in the sample. In one embodiment integrity of the nucleic acid being evaluated is an important feature to permit the distinction between the genomic material from the fetal cells and other cells in the sample. In particular, enrichment and separation of fetal cells using the arrays of obstacles produces gentle treatment which minimizes cellular damage and maximizes nucleic acid integrity permitting exceptional levels of separation and the ability to subsequently utilize various formats to very accurately analyze the genome of the cells which are present in the sample in extremely low numbers.

An enrichment device of the invention can comprise one or more size-based separation modules fluidically coupled upstream to one or more capture modules. The capture modules can be configured to selectively enrich the nRBC's from other larger cells not comprising hemoglobin. For example a capture module can selectively bind cells of interest such as nRBC's. Capture modules can include a substrate having multiple obstacles that restrict the movement of cells or analytes greater than a predetermined size. Examples of capture modules that inhibit the migration of cells based on size are disclosed in U.S. Pat. Nos. 5,837,115 and 6,692,952, which are herein incorporated by reference in their entirety.

In some embodiments a capture module captures analytes (e.g., analytes of interest or not of interest) based on their affinity. For example an affinity-based separation module can include an array of obstacles with binding moieties attached, which selectively bind one or more analytes of interest (e.g., red blood cells, fetal cells, or nRBCs) or analytes not-of-interest (e.g., enucleated RBCS or white blood cells). See, e.g., WO 2007/029221, which is herein incorporated by reference in its entirety. Arrays of obstacles adapted for separation by capture can include obstacles having one or more shapes and can be arranged in a uniform or non-uniform order. In some embodiments, a two-dimensional array of obstacles is staggered such that each subsequent row of obstacles is offset from the previous row of obstacles to increase the number of interactions between the analytes being sorted (separated) and the obstacles.

Binding moieties coupled to the obstacles can include e.g., proteins (e.g., ligands/receptors), nucleic acids having complementary counterparts in retained analytes, antibodies, etc. In some embodiments, an affinity-based separation module comprises a two-dimensional array of obstacles covered with one or more antibodies selected from the group consisting of: anti-CD71, anti-CD45, anti CD-36, anti-GPA and anti-CD34

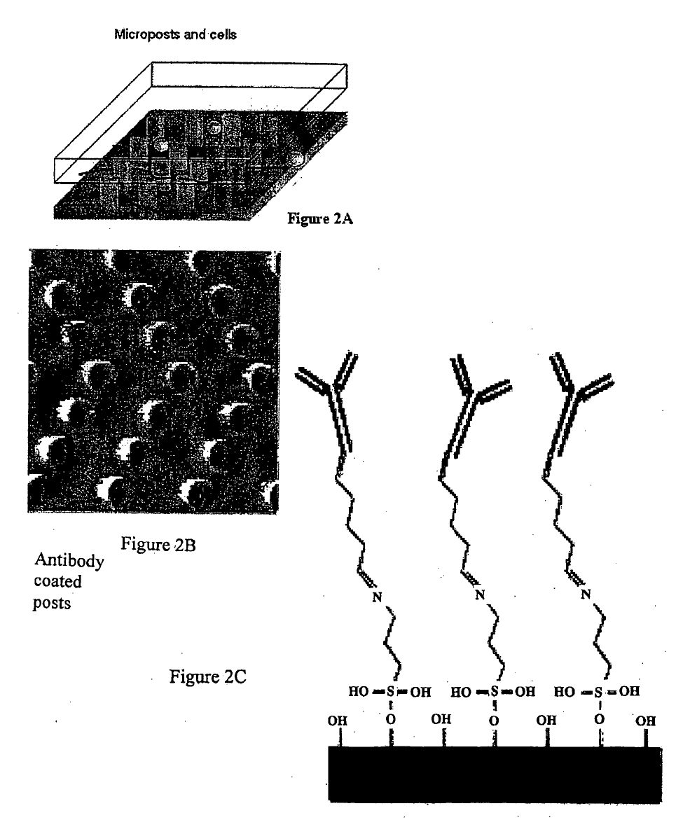

FIG. 2A illustrates a path of a first analyte through an array of posts wherein an analyte that does not specifically bind to a post continues to migrate through the array, while an analyte that does bind a post is captured by the array. FIG. 2B is a picture of antibody coated posts. FIG. 2C illustrates coupling of antibodies to a substrate (e.g., obstacles, side walls, etc.) as contemplated by the present invention. Examples of such affinity-based separation modules are described in WO 2004/029221, which is herein incorporated by reference in its entirety.

In some embodiments, a capture module utilizes a magnetic field to separate and/or enrich one or more analytes (cells) based on a magnetic property or magnetic potential in an analyte. For example, red blood cells which are slightly diamagnetic (repelled by magnetic field) in physiological conditions can be made paramagnetic (attributed by magnetic field) by deoxygenation of the hemoglobin into methemoglobin. This magnetic property can be achieved through physical or chemical treatment of the red blood cells. Cells containing hemoglobin can be enriched by treating them with a reagent to render the cells magnetically responsive. These cells can then be enriched from a mixed population of cells (e.g., a raw blood sample or a size enriched sample) by flowing the sample through a magnetic field (e.g., uniform or non-uniform). In one embodiment the reagent is sodium nitrite.

In one embodiment an enrichment device can have both one or more size based separation module(s) and one or more capture module(s) in series. This allows for a maternal blood sample to flow first through a size-based separation module to remove enucleated cells and cellular components (e.g., analytes having a hydrodynamic size less than 4, 5, or 6 μms) based on size. The size enriched larger cells (e.g., analytes having a hydrodynamic size greater than 4, 5, or 6 μms), such as white blood cells and nucleated red blood cells, can be treated with a reagent, such as CO₂, N₂, Na₂S₂O₄, or NaNO₂, that alters a magnetic property of the red blood cells' hemoglobin. The treated sample can then flow through a micro channel, channel or a column coupled to an external magnet, or a column containing large magnetic obstacles. Paramagnetic analytes (e.g., nucleated red blood cells) can be captured by the magnetic field while white blood cells and other non-red blood cells flow through the magnetic field. This device enriches a sample for nRBCs (including mnRBC's and/or fnRBC's). Additional examples of magnetic separation modules are described in US 2006-0223178 and US 2007-0196820, which are herein incorporated by reference in their entirety.

Other means of rendering cells magnetic include by adsorption of magnetic cations. Paramagnetic cations include, for example, Cr⁺³, Co⁺², Mn⁺², Ni⁺², Fe⁺³, Fe⁺², La⁺³, Cu⁺², GD⁺³, Ce⁺³, Tb⁺³, Pr⁺³, Dy⁺³, Nd⁺³, Ho⁺³, Pm⁺³, Er⁺³, Sm⁺³, Tm⁺³, Fu⁺³, Yb⁺³, and Lu⁺³ (U.S. Patent Application Publication No. 20060078502). For instance, red blood cells can be rendered paramagnetic with chromium by contacting cells with an aqueous solution of chromate ions (Eisenberg et al. U.S. Pat. No. 4,669,481).

Cells may be rendered magnetic by conjugating a magnetic agent to a targeting compound that binds to the cell surface. Suitable targeting compounds include, for example, proteins, antibodies, hormones, and ligands. For example, cells may be rendered magnetic by coating magnetic nanoparticles with strepavidin or avidin; biotinylating the cells, and contacting the cells with the coated nanoparticles (WO/2000/071169). Magnetic agents can also be treated to form magnetodendrimers by any means known in the art, for example the method of Bulte et al., (Magneto Dendrimers as a New Class of Cellular Contrast Agents. Pro. Internat. Soc.).

Supermagnetic iron oxides which may be used in the current invention include (magneto) ferritins, (magneto) liposomes, (magneto) dendrimers, dysprosium, and gadolinium-or-iron-containing macromolecular chelates. The superparamagnetic iron oxide can be magnetic iron oxide nanoparticles (MION), for example, MION-46L. MION-46L is a dextran-coated magnetic nanoparticle with a superparamagnetic maghemite- or magnetite-like inverse spinel core structure.

Additional enrichment steps can be used to separate fnRBC's from mnRBCs. In some embodiments, a sample enriched by size-based separation followed by affinity/magnetic separation is further enriched using fluorescence activated cell sorting (FACS) or selective lysis of a subset of the enriched cells.

In some embodiments, subsequent enrichment involves isolation of rare cells or rare DNA (e.g. fetal cells or fetal DNA) by selectively initiating apoptosis in the cells of interest. This can be accomplished, for example, by subjecting a sample that includes rare cells (e.g. a mixed sample) to hyperbaric pressure (increased levels of CO₂; e.g. 4% CO₂). This selectively initiates condensation and/or apoptosis in the rare or fragile cells in the sample (e.g. fetal cells). Once the rare cells (e.g. fetal cells) begin apoptosis, their nuclei will condense and optionally be ejected from the rare cells. The rare cells or nuclei can be detected using any technique known in the art to detect condensed nuclei, including DNA gel electropheresis, in situ labeling fluorescence labeling, and in situ labeling of DNA nicks using terminal deoxynucleotidyl transferase (TdT)-mediated dUTP in situ nick labeling (TUNEL) (Gavrieli, Y., et al. J. Cell Biol. 119:493-501 (1992)), and ligation of DNA strand breaks having one or two-base 3′ overhangs (Taq polymerase-based in situ ligation; Didenko V., et al. J. Cell Biol. 135:1369-76 (1996)).

In some embodiments ejected nuclei can be detected using a size based separation module adapted to selectively enrich nuclei and other analytes smaller than a predetermined size (e.g. 4, 5, or 6 microns) and isolate them from cells and analytes having a hydrodynamic diameter larger than a predetermined size (e.g. 4, 5, or 6 microns). In one embodiment, the present invention contemplates detecting fetal cells/fetal DNA and optionally using such fetal DNA to diagnose or prognosticate a condition in a fetus. For example detection and diagnosis can occur by obtaining a blood sample from a pregnant female, enriching the sample for cells and analytes larger than 8 microns using, for example, an array of obstacles adapted for size-base separation where the predetermined size of the separation is 8 microns (e.g. the gap between obstacles is up to 8 microns). Then, the enriched product can be further enriched for nRBCs by oxidizing the sample to make the hemoglobin paramagnetic and flowing the sample through one or more magnetic regions. This selectively captures the nRBCs and removes other cells (e.g. white blood cells) from the sample. Subsequently, the fnRBC's can be enriched from mnRBC's in the second enriched product by subjecting the second enriched product to hyperbaric or hypobaric pressure or other stimulus that selectively causes the fetal cells to begin apoptosis and condense/eject their nuclei. Condensed nuclei can then be identified/isolated using e.g. laser capture microdissection or a size based separation module that separates components smaller than 3, 4, 5 or 6 microns from a sample. Such fetal nuclei can then by analyzed using any method known in the art or described herein.

In some embodiments, when the analyte desired to be separated (e.g., red blood cells or white blood cells) is not ferromagnetic or does not have a potential magnetic property, a magnetic particle (e.g., a bead) or compound (e.g., Fe³⁺) can be coupled to the analyte to give it a magnetic property. In some embodiments, a bead coupled to an antibody that selectively binds to an analyte of interest can be decorated with one or more antibodies selected from the group of anti CD-71, anti CD-34, anti CD GPA, anti-CD45, anti-CD36.

In some embodiments a magnetic compound, such as Fe³⁺, can be couple to an antibody such as those described above. The magnetic particles or magnetic antibodies herein can be coupled to any one or more of the devices herein prior to contact with a sample or can be mixed with the sample prior to delivery of the sample to the device(s). Magnetic particles can also be used to decorate one or more analytes (cells of interest or not of interest) to increase the size prior to performing size-based separation.

Magnetic field used to separate analytes/cells in any of the embodiments herein can uniform or non-uniform as well as external or internal to the device(s) herein. An external magnetic field is one whose source is outside a device herein (e.g., container, channel, obstacles). An internal magnetic field is one whose source is within a device contemplated herein. An example of an internal magnetic field is one where magnetic particles can be attached to obstacles present in the device (or manipulated to create obstacles) to increase surface area for analytes to interact with to increase the likelihood of binding. Analytes captured by a magnetic field can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will.

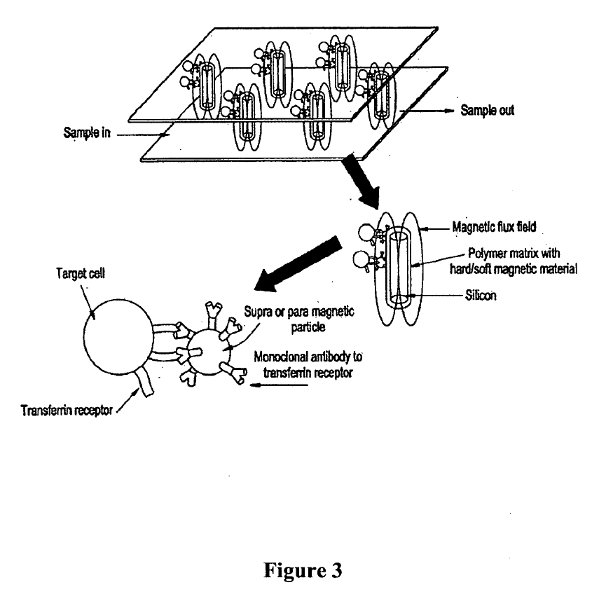

FIG. 3 illustrates an embodiment of a device configured for capture and isolation of cells expressing the transferrin receptor from a complex mixture. Monoclonal antibodies to CD71 receptor are readily available off-the-shelf and can be covalently coupled to magnetic materials, such as, but not limited to any ferroparticles including but not limited to ferrous doped polystyrene and ferroparticles or ferro-colloids (e.g., from Miltenyi and Dynal). The anti CD71 bound to magnetic particles can then be flowed into the device. The antibody coated particles are drawn to the obstacles (e.g., posts), floor, and walls and are retained by the strength of the magnetic field interaction between the particles and the magnetic field. The particles between the obstacles and those loosely retained with the sphere of influence of the local magnetic fields away from the obstacles can be removed by a rinse with a buffer or wash fluid.

In some embodiments, a fluid sample such as a blood sample is first flowed through one or more size-base separation module. These size-base separation modules can be fluidly connected in series and/or in parallel.

In another embodiment waste (e.g., cells having hydrodynamic size less than 4 microns) from a size based separation module can be directed into a first outlet and the product (e.g., cells having hydrodynamic size greater than 4 microns) can be directed to a second outlet. Cells in the product can be subsequently enriched by rendering them magnetically responsive. In one embodiment the product is modified (e.g., by addition of one or more reagents) such that the hemoglobin in the red blood cells becomes paramagnetic. In another embodiment the product is exposed to magnetically responsive beads (e.g., ferrous beads) with cell specific binding moieties (e.g. antibodies). Subsequently, the product is flowed through one or more magnetic fields. The cells that are trapped by the magnetic field can then be analyzed using the one or more methods herein.

One or more of the enrichment modules herein (e.g., size-based separation module(s) and capture module(s)) can be fluidly coupled in series or in parallel with one another. For example a first outlet from a separation module can be fluidly coupled to a capture module. In some embodiments, the separation module and capture module are integrated such that a plurality of obstacles acts both to deflect certain analytes according to size and direct them in a path different than the direction of analyte(s) of interest, and also as a capture module to capture, retain, or bind certain analytes based on size, affinity, magnetism or other physical property.

In any of the embodiments described herein, the enrichment steps performed can have a specificity and/or sensitivity greater than 50, 60, 70, 80, 90, 95, 96, 97, 98, 99, 99.1, 99.2, 99.3, 99.4, 99.5, 99.6, 99.7, 99.8, 99.9 or 99.95% The retention rate of the enrichment module(s) herein is such that ≧50, 60, 70, 80, 90, 91, 92, 93, 94, 95, 96, 97, 98, 99, or 99.9% of the analytes or cells of interest (e.g., nucleated cells or nucleated red blood cells or nucleated from red blood cells) are retained. Simultaneously, the enrichment modules are configured to remove ≧50, 60, 70, 80, 85, 90, 91, 92, 93, 94, 95, 96, 97, 98, 99, or 99.9% of all unwanted analytes (e.g., red blood-platelet enriched cells) from a sample.

For example, in some embodiments the analytes of interest can be retained in an enriched solution that is less than 50, 40, 30, 20, 10, 9.0, 8.0, 7.0, 6.0, 5.0, 4.5, 4.0, 3.5, 3.0, 2.5, 2.0, 1.5, 1.0, or 0.5 fold diluted from the original sample. In some embodiments, any or all of the enrichment steps increase the concentration of the analyte of interest (e.g., nrBCs, mnRTBCs, fnRBCs, fetal cells or trophoblasts), for example, by transferring them from the fluid sample to an enriched fluid sample (sometimes in a new fluid medium, such as a buffer).

### Sample Analysis

In one aspect of the invention a method is disclosed for determining the likelihood of the presence of a condition in a fetus, such as an abnormal condition. In one embodiment the number of nRBCs in a sample from a pregnant female can be determined (such as by counting) and a likelihood of a fetal abnormal condition is determined based on the comparison between statistical averages of nRBCs from samples from pregnant females with normal fetuses with statistical averages of nRBCs from samples from pregnant females with fetuses with an abnormal condition. In another embodiment, the likelihood of the presence of an abnormality in a fetus can be calculated by determining the number of nRBCs in a sample from a mother of the fetus and comparing them to a pre-determined threshold number for nRBCs obtained from samples from mothers with known normal fetuses and/or with mothers with known abnormal fetuses. The biologic fluids that can be sampled and compared include, but are not limited to, blood, amniotic, cervical, or vaginal fluids. In one embodiment the fetal abnormal condition is a genetic abnormality such as an aberration in chromosome number, an error in DNA sequence, an error in methylation status or an error in chromosome imprinting. A fetal abnormal condition can include aneuploidy, segmental aneuploidy, Alpha-1-antitrypsin (A1A) deficiency, Achondroplasia, β-thalassemia, Bloom syndrome, Cystic Fibrosis (CF), Familial Dysautonomia (Riley Day syndrome), Familial Mediterranean Fever (FMF), Fibrodysplasia Ossificans Progressiva (FOP), Hutchinson-Gilford Progeria syndrome, Lesch-Nyhan Syndrome (LNS) & Variant (LNV), Multiple Sclerosis (MS), Polycystic kidney disease (PKD), Tay Sachs, Tuberous sclerosis, Wilson Disease, or Wolman disease.

In another aspect of the invention a method is disclosed for determining the likelihood of the presence of an aneuploidy or segmental aneuploidy in a fetus or assessing an increased risk of aneuploidy or segmental aneuploidy in a fetus. In one embodiment the number of nRBCs in a sample from a mother of is determined (such as by counting) and a likelihood of a fetal aneuploidy is determined based on the comparison between statistical averages of nRBCs from samples from mothers with diploid fetuses compared with statistical averages of nRBCs from samples from mothers with aneuploid fetuses. In another embodiment, the likelihood of the presence of an aneuploidy in a fetus can be calculated by determining the number of nRBCs in a sample from a mother of the fetus and comparing them to pre-determined threshold numbers for nRBCs obtained from samples from mothers with known diploid fetuses and with mothers with known aneuploid fetuses. The biologic fluids that can be sampled and compared include blood, amniotic, cervical, or vaginal fluids.

Aneuploidy means the condition of having less than or more than the normal diploid number of chromosomes. In other words, it is any deviation from euploidy. Aneuploidy includes conditions such as monosomy (the presence of only one chromosome of a pair in a cell's nucleus), trisomy (having three chromosomes of a particular type in a cell's nucleus), tetrasomy (having four chromosomes of a particular type in a cell's nucleus), pentasomy (having five chromosomes of a particular type in a cell's nucleus), triploidy (having three of every chromosome in a cell's nucleus), and tetraploidy (having four of every chromosome in a cell's nucleus). Birth of a live triploid is extraordinarily rare and such individuals are quite abnormal, however triploidy occurs in about 2-3% of all human pregnancies and appears to be a factor in about 15% of all miscarriages. Tetraploidy occurs in approximately 8% of all miscarriages (http://www.emedicine.com/med/topic3241.htm). Segmental aneuploidy means having less than or more than the normal diploid number of chromosomal segments. Examples of segmental aneuploidy include, but are not limited to, 1p36 duplication, dup(17)(p11.2p11.2) syndrome, Pelizaeus-Merzbacher disease, dup(22)(q11.2q11.2) syndrome, and cat-eye syndrome.

An abnormal or aneuploid condition of a fetus or an increased risk for such a condition can be determined when the total number of nRBC's in the sample is greater than 1, 2, 3, 4, 5, 6, 7, 8, 9, 10, 20, 30, 50, 70, 90, 100, 150, 200, 150, 200, 250, 300, 350, 400, 450, 500, 550, 600, 650, 700, 750, 800, 850, 900, 950, or 1,000 nRBC/mL. Sample volume useful in the disclosed methods can range from 10 ml to 100 mL, such as 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 45 ml, 50 ml, 55 ml, 60 ml, 65 ml, 70 ml, 75 ml, 80 ml, 85 ml, 90 ml, 95 ml, or 100 ml. Samples can be obtained from a pregnant woman at first trimester or second trimester.

The presence of a maternal condition can be determined based on enumerating nucleated red blood cells from a sample. Maternal conditions that can be determined include severe infection, hypoxia, pre-eclampsia, diabetes, solid tumors, acute and chronic hematological malignancies, leukemia, myeloproliferative syndromes (e.g. myelosclerosis and caricinomatosis), benign hematological conditions (e.g. hemolysis, hemorrhage, nutritional anaemia, infectious mononucleosis, myelodysplasia, Hb-SS, thalassaemia), septicemia, inflammatory bowel disease, chronic lung disease, fractures, myocardial infarction, and liver disease.

In some embodiments, the biologic sample can be processed in order to enrich for nRBCs relative to enucleated cells prior to enumerating the number of nRBCs present in the sample. nRBCs can be enriched by a variety of methods including, but not limited to, one or more of the following, performed at the same time or in sequence: enrichment based on cell size, affinity selection based on anti-CD45, anti-CD36, anti-GPA, anti-CD71 and anti-CD 34 antibodies, or affinity selection based of nRBCs rendered magnetically responsive. Size based separation can involve, for example, flowing a maternal sample mother through a microfluidic device that selectively directs cells and particles larger than a certain size to a first outlet and cells or particles smaller than a certain size to a second outlet. This can enrich nucleated cells (e.g., nRBCs) relative to non-nucleated cells (such as enucleated red blood cells). The nRBCs can also be enriched relative to nucleated cells (such as white blood cells). In one embodiment this can be accomplished using, affinity selection, whereby white blood cells are selected using an antibody that selectively binds white blood cells relative to red blood cells. In another embodiment this can also be accomplished using magnetic separation. For example, nRBCs can be rendered magnetically responsive by treating them with a reagent that alters a magnetic property of the cells, such as by altering the oxidation or reduction state of the hemoglobin in said cells. In another embodiment the magnetic beads can be bound to nRBCs for affinity selection.

In one embodiment, magnetic separation involves adding a reagent that alters the magnetic property of hemoglobin, e.g., sodium nitrite oxidation of hemoglobin to methamoglobin. This renders the hemoglobin containing red blood cells magnetically responsive. In the presence of a magnetic field, the red blood cells can be separated from the white blood cells. Thus, an affinity separation following a size-based separation can be used to enrich nucleated red blood cells from a sample. In any of the embodiments disclosed herein, the cells can be lysed in a way such that the nuclei of the cells remain intact. In some embodiments, lysing occurs prior to enumerating the number of nRBCs present.

The systems and methods herein can further be utilized for performing association studies. For example, in some embodiments, the systems and methods herein are used to perform association studies based on data collected from a plurality of control samples and a plurality of case samples. For example, fluid samples (e.g., blood samples) can be collected from more than 10, 20, 50, or 100 case individuals (individuals with a phenotypic condition) and from more than 10, 20, 50, or 100 control individuals (those not inhibiting the phenotypic condition). Samples from each individual can then be enriched for a first or a plurality of analytes (e.g., nRBCs, mnRBCs, fnRBCs or trophoblasts). Such analytes can then be enumerated and/or characterized and data collected. This data can be subsequently be used to perform an association study. Data can be stored in an electronic database. The association study can be performed using a computer executable logic for identifying one or more characteristics associated with case or control samples. For example an association study between the number of nRBCs in a sample and a specific fetal abnormal condition can be used to develop a diagnostic or prognostic test. In one embodiment the system is an analyzer system.

In one embodiment, fluid samples obtained from individuals for an association study are blood samples. The analytes (such as nucleated red blood cells) enriched from such samples can be ones that have a hydrodynamic size greater than 4 microns, or greater than 6, 8, 10, 12, 14, or 16 microns. In some embodiments, samples obtained from individuals are enriched for one or more cells selected from the group consisting of: a RBC, a fetal RBC, a trophoblast, a fetal fibroblast, a white blood cell (WBCs), an infected WBC, a stem cell, an epithelial cell, an endothelial cell, an endometrial cell, a progenitor cell. In one embodiment the cells that are enriched are those that are found in vivo at a concentration of less than 1×10⁻¹, 1×10⁻², or 1×10⁻³ cells/IL. In another embodiment the cells can be at least 99% of the cells of interest (those enriched) from the sample are retained. Enrichment for purposes of conducting an association study can increase the concentration of a first cell type of interest by at least 10,000 fold.

The enriched analytes (e.g. nucleated red blood cells) can then be analyzed to determine one or more characteristics. Such characteristics can include, e.g., the presence or absence of an analyte in a sample, quantity of an analyte, ratio of two analytes (e.g., endothelial cells and epithelial cells), morphology of one or more analytes, genotype of analyte, proteome of analyte, RNA composition of analyte, gene expression within an analyte, microRNA levels, or other characteristic traits of the analytes enriched are subsequently used to perform an association study.

In some embodiments, an analyzer system can be configured to perform an analysis step such as detecting, enumerating, or analyzing analytes of interest, e.g., nucleated red blood cells (mnRBCs or fnRBCs), trophoblasts or cell fragments (such as a nucleus or a chromosome). An analyzer system comprises an analyzer and, optionally, at least one of a computer, a monitor and a command interface (e.g., a keyboard, mouse, trackball or joystick). Exemplary analyzers include, but are not limited to, a cell counter, a fluorescent activated cell sorting (FACS) machine, or a microscope. The number of analytes of interest (such as mnRBCs or fnRBCs) detected in a sample can be used by the analyzer or a user to determine a diagnosis or prognosis of a fetal condition such as an abnormal condition. In some embodiments, an analyzer system compares (and optionally stores) data collected with known data points. In some embodiments, an analyzer system compares (and optionally stores) data collected from case samples and control samples and performs an association study. For example an analyzer system can compare the statistical averages of nRBCs from samples from mothers with normal fetuses with statistical averages of nRBCs from samples from mothers with abnormal fetuses. This comparison can be used to determine a threshold value which can be used to determine a diagnosis or prognosis based on the results obtained for a subject of interest (e.g. a pregnant female)

In some embodiments, an analyzer system comprises a computer executable logic that detects a probe signal from one or more probes that selectively bind an enriched analyte of interest, or components thereof. In some embodiments, the computer executable logic also analyzes such signals for their intensity, size, shape, aspect ratio, and/or distribution. The computer executable logic can then general a call based on results of analyzing the probe signals.

Examples of probes whose signals can be detected/analyzed by an analyzer include, but are not limited to, a fluorescent probe (e.g., for staining chromosomes such as X, Y, 13, 18 and 21 in fetal cells), a chromogenic probe, a direct immunoagent (e.g. labeled primary antibody), an indirect immunoagent (e.g., unlabeled primary antibody coupled to a secondary enzyme), a quantum dot, a fluorescent nucleic acid stain (such as DAPI, Ethidium bromide, Sybr green, Sybr gold, Sybr blue, Ribogreen, Picogreen, YoPro-1, YoPro-2 YoPro-3, YOYo, Oligreen acridine orange, thiazole orange, propidium iodine, or Hoeste), another probe that emits a photon, or a radioactive probe. In some embodiments, an analyzer can detect a chromogenic probe, which can provide a faster read time than a fluorescent probe. In some embodiments, an analyzer comprises a computer executable logic that performs karyotyping, in situ hybridization (ISH) (e.g., florescence in situ hybridization (FISH), chromogenic in situ hybridization (CISH), nanogold in situ hybridization (NISH)), restriction fragment length polymorphism (RFLP) analysis, polymerase chain reaction (PCR) techniques, flow cytometry, electron microscopy, quantum dot analysis, or detects single nucleotide polymorphisms (SNPs) or levels of RNA. In some embodiments, two or more probes are used, which can emit different wavelengths. For example, multiple FISH probes or other DNA probes can be used in analyzing a cell or component of interest. Methods for using FISH to detect rare cells are disclosed in Zhen, D. K., et al. (1999) Prenatal Diagnosis, 18(11), 1181-1185, Cheung, MC., (1996) Nature Genetics 14, 264-268, which are incorporated herein by reference for all purposes. Methods for using CISH are disclosed in Amould, L. et al British Journal of Cancer (2003) 88, 1587-1591; and US 2002/0019001, which are incorporated herein by reference in their entirety.

For example, when analyzing nucleated red blood cells enriched from maternal blood, an analyzer can be configured to detect nucleated red blood cells or components thereof. In some embodiments, analysis of fetal cells (such as fnRBCs) or components thereof is used to determine the sex of a fetus; the presence/absence of a genetic abnormality (e.g., chromosomal, DNA or RNA abnormality); or one or more SNPs. In one embodiment an analyzer uses flow cytometry to enumerate the number of cells (nucleated red blood cells, mnRBCs, fnRBCs or trophoblasts) enriched from a maternal blood sample.

Flow cytometry generally uses an apparatus that comprises a beam of light (usually laser light) of a single wavelength that is directed onto a hydro-dynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals found in the particle or attached to the particle can be excited into emitting light at a lower frequency than the light source. This combination of scattered and fluorescent light is picked up by the detectors, and by analysing fluctuations in brightness at each detector (one for each fluorescent emission peak) it is then possible to derive various types of information about the physical and chemical structure of each individual particle. FSC correlates with the cell volume and SSC depends on the inner complexity of the particle (i.e. shape of the nucleus, the amount and type of cytoplasmic granules or the membrane roughness).

The nRBCs from the test sample can be imaged prior to, during or after enumeration. Likewise, FISH can be performed on the nucleated red blood cells prior to, during or after enumeration.

The results of the enumeration step of nRBCs or aneuploid nRBCs can be compared to threshold or pre-determined values to determine if there is an increased likelihood of certain genetic characteristics of the fetus. For example, if the number of nRBCs in a peripheral blood sample from a pregnant female exceeds a threshold value then an increased likelihood of an abnormal fetal condition can be determined.

Fetal conditions in which the likelihood of occurrence can be calculated include abnormal conditions such as aneuploidy and segmental aneuploidy, for example: trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY or XYYYY. Other abnormal conditions where the likelihood of occurrence can be calculated include Klinefelter Syndrome, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), and 1p36 deletion and any combination of the above.

In one embodiment, the fetal abnormal condition to be detected is due to one or more deletions in a sex or autosomal chromosome, for example: Cri-du-chat syndrome, Wolf-Hirschhorn syndrome, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, steroid sulfatase deficiency, Kallmann syndrome, Microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c) and 1p36 deletion. In some embodiments, the fetal abnormal condition is an abnormal decrease in chromosomal number, such as XO syndrome.

In another aspect of the invention, a condition in a fetus in a subject can be determined by enriching one or more nucleated red blood cells from a biologic sample obtained from said subject and determining a condition of the fetus based on the number of nucleated red blood cells isolated from the biologic sample. Sources of biologic samples include blood, amniotic fluid, and cervical swabs.

In one embodiment a method is used for determining the likelihood or increased likelihood of the presence of an abnormal condition in a fetus by determining the number of nucleated red blood cells (nRBCs) as well as performing one or more maternal serum marker screens, and using the combined results to assign a likelihood of the fetus being abnormal. This can be based on statistical averages of nRBCs and corresponding maternal serum marker screening results from samples from mothers with abnormal fetuses. In another embodiment, the likelihood of the presence of an abnormal condition in a fetus can be determined by enumerating the number of nRBCs and results from maternal serum marker screens in a sample from the mother of the fetus and comparing them to threshold number of nRBCs and maternal serum marker levels. The biologic fluids that can be sampled and compared include blood, amniotic, cervical, or vaginal fluids.

In another aspect of the invention, a condition of a fetus in a subject can be determined by enriching one or more nucleated red blood cells from a biologic sample obtained from said subject and combining this with the detection of serum markers and/or using diagnostic ultrasound to measure space in nuchal fold of the fetus, and determining a condition of the fetus based on the number of nucleated red blood cells isolated and the presence and/or concentration of the serum markers.

Examples of maternal serum marker screening include but are not limited to alpha-fetoprotein (AFP), maternal serum alpha-fetoprotein (MSAFP), Double Marker Screen, Double Screen, Triple Marker Screen, Triple Screen, Quad Screen, Quad Marker Screen, Penta Screen, Penta Marker Screen, 1^{st} Trimester Screen, 2^{nd} Trimester Screen, Integrated Screen, Combined Screen, Contingency Screen, Repeated Measures Screen or Sequential Screen. The specific serum markers include but are not limited to Pregnancy Associated Plasma Protein-A (papA), free β HCG, unconjugated estriol (UE3), alpha-fetoprotein (AFP), human Chorionic Gonadotropin (HCG), inhibin, D-inhibin A (DIA), and Invasive Trophoblast Antigen (ITA, or hhCG). The Double Screen (a.k.a. Double Marker Screen) usually uses AFP and hCG as markers. The Triple Screen (a.k.a. Triple Marker Screen) usually uses AFP, hCG, and uE3 as markers. The Quad Screen (a.k.a. Quad Marker Screen) usually uses AFP, hCG, uE3, and inhibin as markers. The Penta Screen (a.k.a. Penta Marker Screen) uses AFP, hCG, uE3, inhibin, and ITA as markers. In some embodiments, a Nuchal Translucency (NT) test is used in combination with enumeration of nRBC's and optionally detection of serum markers to determine a more accurate diagnosis of fetal abnormal condition (such as fetal aneuploidy). In some embodiments, the results are correlated with the Mother's age, for example with whether or not a human mother is under the age of 35. The 1^{st} Trimester Screen includes the use of papA, free β HCG, ITA, and an NT test individually or in combination (the combination of papA, free a HCG, and NT is sometimes referred to as the Combined Screen). The 2^{nd} Trimester Screen includes the use of AFP, hCG, uE3, DIA, and ITA individually or in combination. The Integrated Screen includes the use of papA and NT in the 1^{st} trimester, and combines the results with a Quad Screen (AFP, hCG, uE3, inhibin) in the 2^{nd} trimester. The Sequential Screen comprises a 1^{st} Trimester Screen followed by a Quad Screen plus papA and NT in the 2^{nd} trimester. The Contingency Screen is a staged screen, including a 1^{st} Trimester Screen, followed if necessary by a Quad Screen. The Sequential Screen includes the Integrated Screen followed by a 2^{nd} Trimester Screen. The Repeated Measures Screen includes measuring a serum marker such as papA in the 1^{st} and 2^{nd} trimesters.

The conditions that can be diagnosed include aneuploidy and segmental aneuploidy, such as trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY, XYYYY, Klinefelter Syndrome, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p36 deletion, or a combination thereof.

In another aspect of the invention a business performs an association study to link the number of nucleated red blood cells in a biological sample with various conditions. In some embodiments, the condition is fetal abnormality or fetal aneuploidy. In one embodiment the business can perform the assays necessary to enumerate nRBC's in the sample. In a further embodiment the business can provide a screen based on the enumerated nRBC's. In another embodiment the business can provide a screen based on the combination of enumerated nRBC's and the results of a diagnostic ultrasound and/or serum marker tests. Such serum marker tests can include alpha-fetoprotein (AFP), maternal serum alpha-fetoprotein (MSAFP), Double Marker Screen, Double Screen, Triple Marker Screen, Triple Screen, Quad Screen, Quad Marker Screen, Penta Screen, Penta Marker Screen, 1^{st} Trimester Screen, 2^{nd} Trimester Screen, Integrated Screen, Combined Screen, Contingency Screen, Repeated Measures Screen or Sequential Screen. Such serum markers could include Pregnancy Associated Plasma Protein-A (papA), free β HCG, and Invasive Trophoblast Antigen (ITA) for the 1^{st} trimester, and/or unconjugated estriol (UE3), alpha-fetoprotein (AFP), human Chorionic Gonadotropin (HCG), inhibin, and/or D-inhibin A (DIA) for the 2^{nd} trimester. This combination would provide a high sensitivity and specificity assessment of fetal health. In another embodiment, the clinical service provider conducts fetal testing in regional or localized free-standing facilities or alternately, on-site at hospitals or at physician offices. In a further embodiment, the clinical service provider can be mobile and can be scheduled to perform the testing services on-site at pre-established times or on call. The clinical service providers include CLIA certified laboratories.

In any of the embodiments herein, a confirmation step can also be included. The confirmation step can confirm (i) the presence of fetal cells in the sample, and/or (ii) a fetal abnormal condition y.

In one embodiment, a confirmation step can comprise performing one or more assay on the enriched nRBCs,

for example: fluorescent in-situ hybridization (FISH), polymerase chain reaction (PCR), quantitative polymerase chain reaction (qPCR), nucleic acid analysis such as high-throughput sequencing, SNP detection, RNA expression analysis, or comparative genomic hybridization (CGH) array analysis. In another embodiment, enriched product is binned into a microtiter plate such that statistically each well has only 1 or 0 fetal cells. qPCR can then be performed on individual wells to detect the presence of a Y chromosome (e.g., using a DYZ probe, SRY probe or any other probe specific for the Y chromosome). In another embodiment, FISH probes are applied to the enriched product to detect sex chromosomes X and Y. Cells that are potential male fetus cells (express Y chromosome) are then microdissected and can be further analyzed using qPCR for the Y chromosome. In some embodiments, the enriched cells can flow through a FACS sorter and fetal cells can be identified using probes that are specific to fetal cells or fetal hemoglobin. Examples of fetal specific probes include CD34, and antibodies to fetal globins such as epsilon and gamma. Confirmation can also be accomplished by binning the enriched cells and then determining the levels of expression (mRNA) of various globins such as epsilon, gamma, and beta globins in each well.

Binning may comprise distribution of enriched cells across wells in a plate (such as a 96 or 384 well plate), microencapsulation of cells in droplets that are separated in an emulsion, or by introduction of cells into microarrays of nanofluidic bins. Fetal cells are then identified using methods that may comprise the use of biomarkers (such as fetal (gamma) hemoglobin), allele-specific SNP panels that could detect fetal genome DNA, detection of differentially expressed maternal and fetal transcripts (such as Affymetrix chips), or primers and probes directed to fetal specific loci (such as the multi-repeat DYZ locus on the Y-chromosome). Binning sites that contain fetal cells are then be analyzed for aneuploidy and/or other genetic defects using a technique such as CGH array detection, ultra deep sequencing (such as Solexa, 454, or mass spectrometry), STR analysis, or SNP detection.

Enriched target cells (e.g., nRBC, mnRBC or fnRBC) may be “binned” prior to further analysis of the enriched cells. Binning is any process which results in the reduction of complexity and/or total cell number of the enriched cell output. Binning may be performed by any method known in the art or described herein. One method of binning is by serial dilution. Such dilution may be carried out using any appropriate platform (e.g., PCR wells, microtiter plates) and appropriate buffers. Other methods include nanofluidic systems which can separate samples into droplets (e.g., BioTrove, Raindance, Fluidigm). Such nanofluidic systems may result in the presence of a single cell present in a nanodroplet.

Binning may be preceded by positive selection for target cells including, but not limited to, affinity binding (e.g. using anti-CD71 antibodies). Alternately, negative selection of non-target cells may precede binning. For example, output from a size-based separation module may be passed through a magnetic hemoglobin enrichment module (MHEM) which selectively removes WBCs from the enriched sample by attracting magnetized hemoglobin-containing cells.

For example, the possible cellular content of output from enriched maternal blood which has been passed through a size-based separation module (with or without further enrichment by passing the enriched sample through a MHEM) may consist of: 1) approximately 20 fnRBC; 2) 1,500 fnRBC; 3) 4,000-40,000 WBC; 4) 15×10⁶ RBC. If this sample is separated into 100 bins (PCR wells or other acceptable binning platform), each bin would be expected to contain: 1) 80 negative bins and 20 bins positive for one fnRBC; 2) 150 mnRBC; 3) 400-4,000 WBC; 4) 15×10⁴ RBC. If separated into 10,000 bins, each bin would be expected to contain: 1) 9,980 negative bins and 20 bins positive for one fnRBC; 2) 8,500 negative bins and 1,500 bins positive for one mnRBC; 3) <1-4 WBC; 4) 15×10² RBC. One of skill in the art will recognize that the number of bins may be increased or decreased depending on experimental design and/or the platform used for binning. Reduced complexity of the binned cell populations may facilitate further genetic and/or cellular analysis of the target cells by reducing the number of non-target cells in an individual bin.

Analysis may be performed on individual bins to confirm the presence of target cells (e.g. nRBC, mnRBC or fnRBC) in the individual bin. Such analysis may consist of any method known in the art including, but not limited to, FISH, PCR, STR detection, SNP analysis, biomarker detection, and sequence analysis.

For example, a peripheral maternal venous blood sample enriched by the methods herein can be analyzed to determine pregnancy or a condition of a fetus (e.g., sex of fetus or aneuploidy). The analysis step for fetal cells may further involve comparing the ratio of maternal to paternal genomic DNA on the identified fetal cells.

Any of the techniques herein can be used for prenatal as well as postnatal diagnosis as the fetal cells remain in circulation for a period of time after delivery of the fetus.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

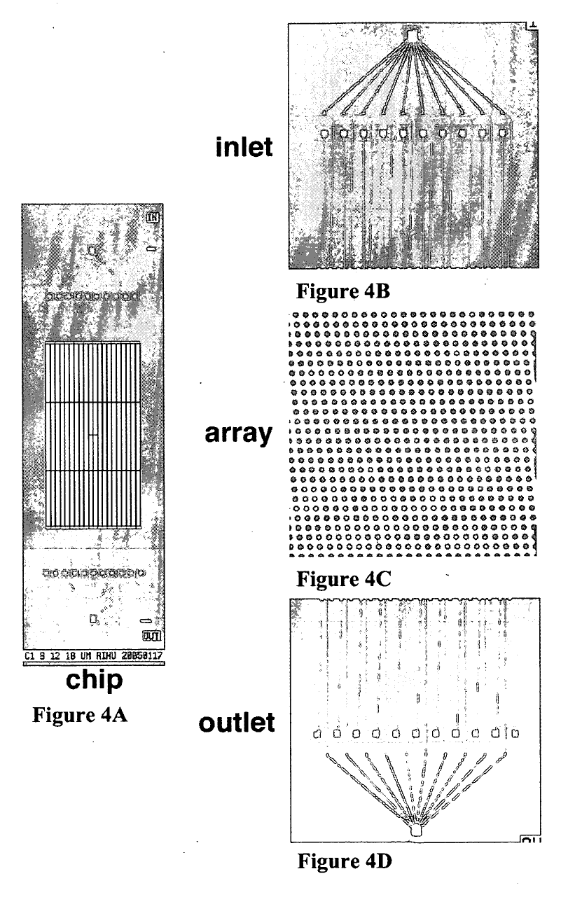

FIGS. 4A-4D shows a schematic of the device used to separate nucleated cells from maternal blood.

Dimensions: 64 mm×32 mm×1 mm

Array design: 1 stage, gap size=20 μm.

Device fabrication: The arrays and channels were fabricated in silicon using standard photolithography and deep silicon reactive etching techniques. The etch depth is 150 μm. Through holes for fluid access are made using KOH wet etching. The silicon substrate was anodically bonded on the etched face to form enclosed fluidic channels with a glass piece (9795, 3M, St Paul, Minn.).

Device packaging: The device was mechanically mated to a plastic manifold with external fluidic reservoirs to deliver blood and buffer to the device and extract the generated fractions.

Device operation: An external pressure source was used to apply a pressure of 1.0 PSI to the buffer and blood reservoirs to modulate fluidic delivery and extraction from the packaged device. The buffer used consists of 1% BSA with 2 mM EDTA in Dulbecco's Phosphate Buffer (iDPBS).

### Example 2

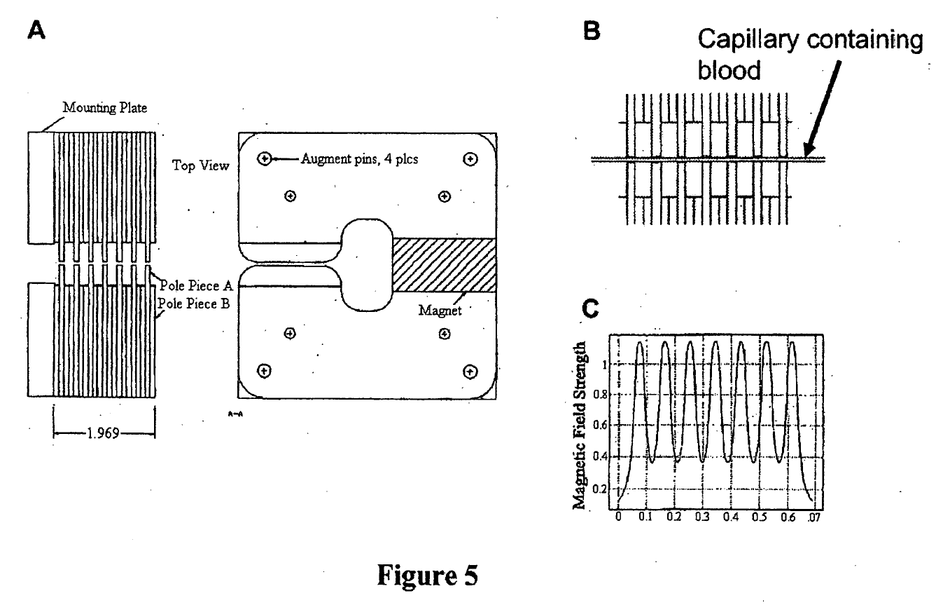

FIGS. 5A and 5B show a schematic of the magnetic separation module used to separate hemoglobin-containing cells from non-hemoglobin-containing cells. This process helps to further separate nucleated cells from maternal blood after enrichment using the process described in Example 1. FIG. 5C is a graph of the field strength of the magnet as a function of the position of the capillary.

Dimensions: 75 mm×13 mm

Device fabrication: A 1.4 Tesla magnet was placed around a Miltenyi LS Column (P/N 130-042401).

Device operation: Prior to device operation, the sample is centrifuged for 10 minutes at 300 g. The sample is then treated with sodium nitrite at 50M for 10 min. The nucleated cells are then passed through the magnetic column (the magnetic separation module) where nucleated red blood cells are retained. In the column, the magnetic field strength is about 1 Tesla, the magnetic field gradient is about 3000 Tesla/m, and the flow velocity is about 0.4 mm/sec. White blood cells are rinsed out of the column using Dulbecco PBS buffer with 1% BSA and 2 μM EDTA, and collected as the negative fraction. The nucleated red blood cells are eluted from the column using the same buffer at a flow velocity of 4 mm/s and collected as the positive fraction.

An external pressure source was used to apply a pressure of 1.4 PSI to the buffer and sample reservoirs to modulate fluidic delivery and extraction from the packaged device.

### Example 3

### Isolation of Fetal Cells from Maternal blood

The device and process described in detail in Example 1 was used in combination with magnetic affinity enrichment techniques to isolate fetal cells from maternal blood.

Experimental conditions: blood from consenting maternal donors carrying male fetuses was collected into K₂EDTA vacutainers (366643, Becton Dickinson, Franklin Lakes, N.J.) immediately following elective termination of pregnancy. The undiluted blood was processed using the device described in Example 1 at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 mL, Sigma-Aldrich, St Louis, Mo.). Subsequently, the nucleated cell fraction was labeled with anti-CD71 microbeads (130-046-201, Mittenyi Biotech Inc., Auburn, Calif.) and enriched using the MiniMACS™ MS column (130-042-201, Miltenyi Biotech Inc., Auburn, Calif.) according to the manufacturer's specifications. Finally, the CD71-positive fraction was spotted onto glass slides.

Measurement techniques: Spotted slides were stained using fluorescence in situ hybridization (FISH) techniques according to the manufacturer's specifications using Vysis probes (Abbott Laboratories, Downer's Grove, Ill.). Samples were stained from the presence of X and Y chromosomes. In one case, a sample prepared from a known Trisomy 21 pregnancy was also stained for chromosome 21.

### Example 4

### Confirmation of the Presence of Male Fetal Cells in Enriched Samples

Confirmation of the presence of a male fetal cell in an enriched sample is performed using qPCR with primers specific for DYZ, a marker repeated in high copy number on the Y chromosome. After enrichment of fnRBC by any of the methods described herein, the resulting enriched fnRBC can be binned by dividing the sample into multiple, i.e. 100 PCR wells. Prior to binning, enriched samples can be screened by FISH to determine the presence of any fnRBC containing an aneuploidy of interest. Because of the low number of fnRBC in maternal blood, only a portion of the wells will contain a single fnRBC (the other wells are expected to be negative for fnRBC). The cells are fixed in 2% Paraformaldehyde and stored at 4° C. Cells in each bin are pelleted and resuspended in 5 μl PBS plus 1 μl 20 mg/ml Proteinase K (Sigma #P-2308). Cells are lysed by incubation at 65° C. for 60 minutes followed by inactivation of the Proteinase K by incubation for 15 minutes at 95° C. For each reaction, primer sets (DYZ forward primer TCGAGTGCATTCCATTCCG; DYZ reverse primer ATGGAATGGCATCAAACGGAA; and DYZ Taqman Probe 6FAM-TGGCTGTCCATTCCA-MGBNFQ), TaqMan Universal PCR master mix, No AmpErase and water are added. The samples are run and analysis is performed on an ABI 7300: 2 minutes at 50° C., 10 minutes 95° C. followed by 40 cycles of 95° C. (15 seconds) and 60° C. (1 minute). Following confirmation of the presence of male fetal cells, further analysis of bins containing fnRBC is performed. Positive bins can be pooled prior to further analysis.

### Example 5

### Clinical Study of Device and Methodology in Subjects with Confirmed Normal or Aneuploidy Fetuses

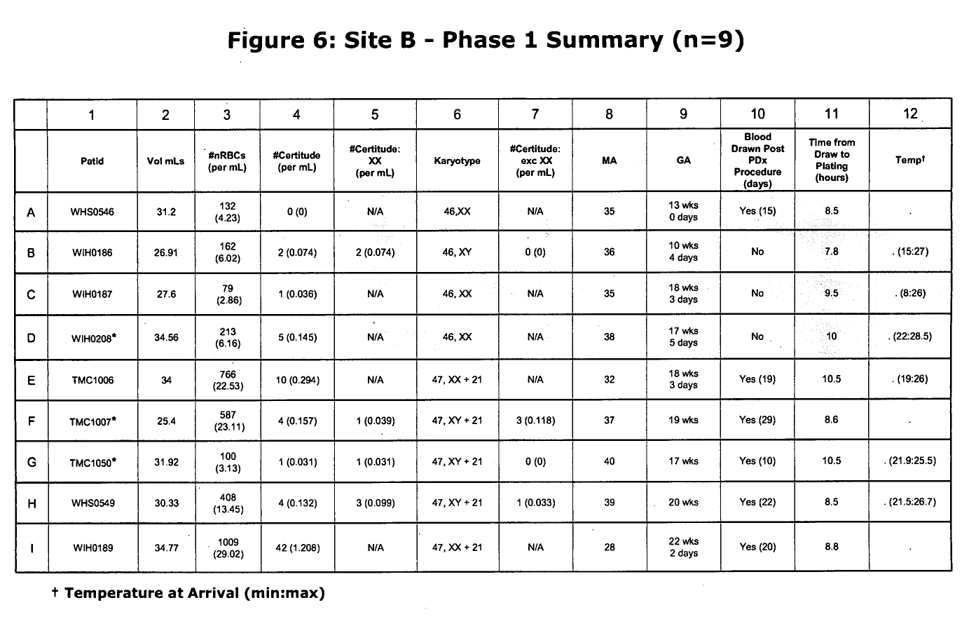

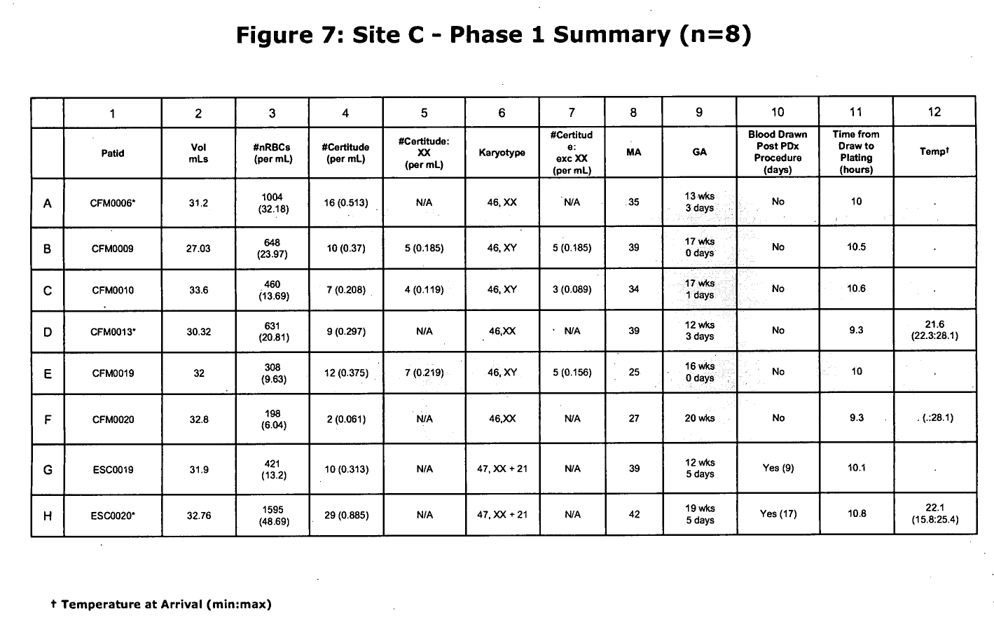

FIGS. 6 and 7 provide a summary of the results of a study performed at Sites B and C respectively, on the blood obtained from four women with normal fetuses and from five women with aneuploidy fetuses. Column 1 lists the subject identification numbers. In column 2 the total volume of blood obtain for the studies is listed. Column 3 lists the total number of nRBCs obtained from the blood along with the number of nRBCs per ml, disclosed in the parenthesizes. Column 4 lists the total Certitude number and Certitude number per ml of blood drawn. In column 5, the Certitude number for XX is disclosed along with the Certitude number for XX per ml of blood. Column 6 lists the confirmed karyotype of the fetus. In column 7, the Certitude number corrected for the presence of XX cells is provided along with the corrected number per ml of blood. Column 8 lists the mother's age, while in column 9 the gestational age is provided. The date of the blood draw post the performance of the subject diagnosis is given in column 10. Column 11 lists the interval in hours between the blood draw and the time the cells were plated. Column 12 lists the temperature of the blood sample on arrival at the analysis facility.

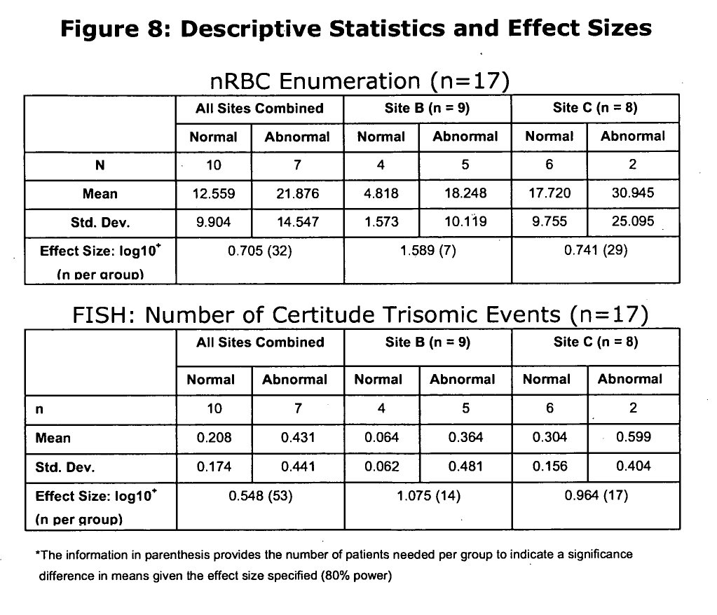

FIG. 8 lists the means and standard deviations for the results of the clinical studies for Sites B and C from FIGS. 6 and 7 for nRBCs enumration and FISH analysis. For nRBC enumeration blood from a total of 17 women was analyzed with 10 carrying normal fetuses and 7 with fetuses with an abnormal condition (ie, abnormal fetuses). The mean number of nRBCs per ml for women with normal fetuses was 12.6, while women with abnormal fetuses the mean number was 22.9. The standard deviations on these values were respectively 9.9 and 14.5. For FISH blood cells from a total of 17 women were analyzed with 10 carrying normal fetuses and 7 with abnormal fetuses. The women with normal fetuses had a mean value of 0.208 fetal cells per ml, while the women with abnormal fetuses had 0.431 fetal cells per ml. Therefore, in one aspect the present invention contemplates measuring total nRBC's in a sample to provide information on a fetal abnormal condition. Due to high standard deviations and low sample size, further testing is required.

### Example 6

### Further Clinical Study of Device and Methodology in Subjects with Confirmed Normal or Aneuploidy Fetuses

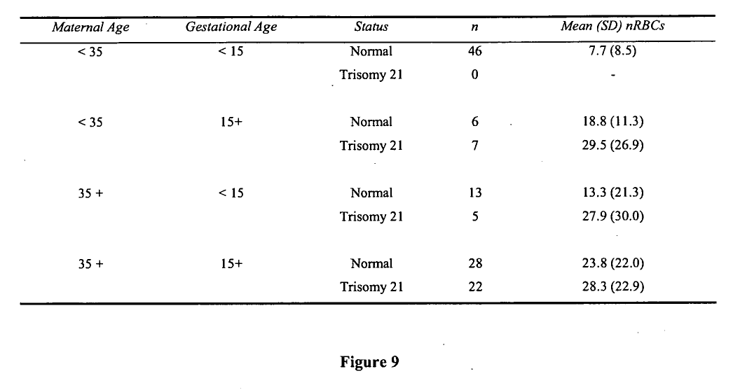

FIG. 9 lists the means and standard deviations for results of clinical studies for nRBC enumeration. A total of 127 women were analyzed with 93 carrying normal fetuses and 34 with abnormal fetuses. The mean and standard deviation (SD) of number of nRBCs for women with normal fetuses, a gestational age of less than 15 weeks, and a maternal age of less than 35 was 7.7 and 8.5 respectively. The mean and SD for women with normal fetuses, a gestational age of 15 or more weeks, and a maternal age of less than 35 was 18.8 and 11.3 respectively. The mean and SD for women with abnormal fetuses, a gestational age of 15 or more weeks, and a maternal age of less than 35 was 29.5 and 26.9 respectively. The mean and SD for women with normal fetuses, a gestational age of less than 15 weeks, and a maternal age of 35 or more was 13.3 and 21.3 respectively. The mean and SD for women with abnormal fetuses, a gestational age of less than 15 weeks, and a maternal age of 35 or more was 27.9 and 30.0 respectively. The mean and SD for women with normal fetuses, a gestational age of 15 or more weeks, and a maternal age of 35 or more was 23.8 and 22.0 respectively. The mean and SD for women with abnormal fetuses, a gestational age of 15 or more weeks, and a maternal age of 35 or more was 28.3 and 22.9 respectively. There were no samples in the abnormal fetus, a gestational age less than 15, and maternal age of less than 35 category.

### Example 7

### Simulation Study Using the FaSTER Trial Data Set to Demonstrate Possible Higher Sensitivity and Specificity Generated from Combining nRBC Enumeration with Maternal Serum Marker Screen Results

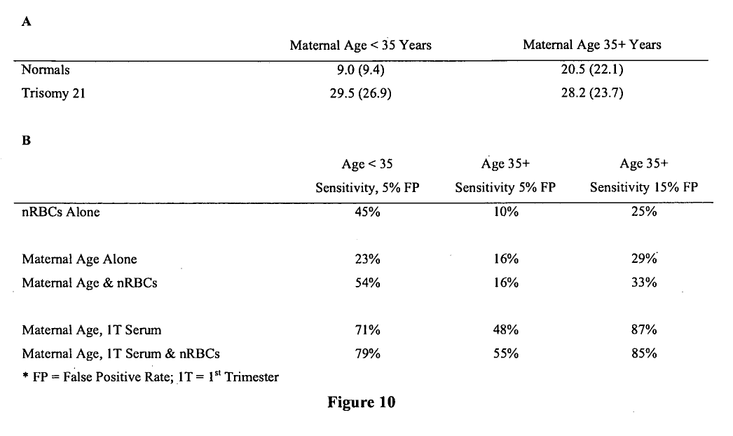

In order to gauge the future usefulness of combining nRBC enumeration with results from maternal serum marker screens for diagnosis of fetal abnormality, a simulation study is performed. The FaSTER (First and Second Trimester Evaluation of Risk) trial data set is a large, national, multicenter study in which numerous woman around the United States were tested using first and second-trimester screening methods for the prenatal detection of Down syndrome (Am J Obstet Gynecol. 2004 October; 191(4): 1446-51). A simulation is designed to compare maternal age, serum markers and nRBC alone and in combination as risk predictors for Down Syndrome. Cases are selected from the FaSTER data set, and nRBCs values are assigned to normals and trisomy 21 cases assuming normal distributions and using means and standard deviations estimated from data shown in FIG. 10A. The data that is used is limited to the subset of subjects with adequate serum screen data.

FIG. 10B shows the sensitivities at a 5% false positive fraction in women <35 years of age and the sensitivities at a 5% and 15% false positive fraction in women 35 years and older. Note that among older women, the false positive fractions are shown to be higher than those observed among younger women. These simulations indicate that nRBC can substantially improve the sensitivity of tests for women under age 35, for a fixed false positive rate of 5%. The addition of nRBC improves sensitivity from 23% to 54% for a test based on maternal age alone. The addition of nRBC improves sensitivity from 71% to 79% for a test based on maternal age and first trimester (IT) serum markers. Based on the results of the initial Artemis study, there appears to be an opportunity to improve on the performance of currently used screening tests.

What is claimed is:

1. A method for determining the presence of a fetal abnormal condition comprising:
enumerating nucleated red blood cells in a blood sample from a pregnant woman; and
determining the presence of a fetal abnormal condition based on the number of nucleated red blood cells in the blood sample.

2. A method for determining the presence of aneuploidy in a fetus, comprising:
a) enumerating nucleated red blood cells in a sample from a pregnant woman;
b) assigning a likelihood of said pregnant woman's fetus being aneuploid based on statistical averages of nucleated red blood cells from blood samples from pregnant women carrying euploid fetuses compared with statistical averages of nucleated red blood cells from blood samples from pregnant women carrying aneuploid fetuses

3. A method for determining the presence of a fetal abnormal condition comprising:
(a) enumerating nRBCs in a first blood sample from a pregnant woman;
(b) and either:
(i) detecting the presence or level of one or more serum markers in the first or a second blood sample from the pregnant woman,
(ii) measuring space in nuchal fold of her fetus; or
(iii) or both (i) and (ii); and
determining the presence of the fetal abnormal condition in the fetus from results from steps (a) and (b).

4. The method of claim 1, 2 or 3, further comprising the step of enriching nucleated red blood cells from enucleated red blood cells or white blood cells.

5. The method of claim 4, wherein said enriching is based on cell size and/or magnetic property.

6. The method of claim 5, wherein said enriching comprises using arrays of obstacles.

7. The method of claim 5, wherein said enriching comprises rendering nucleated red blood cells magnetic.

8. The method of claim 5, wherein said enriching comprises using arrays of obstacles and rendering nucleated red blood cells magnetic.

9. The method of claim 1, 2, or 3, wherein said sample is taken in the first trimester of pregnancy.

10. The method of claim 1, 2 or 3 wherein said pregnant woman is under the age of 35.

11. The method of claim 4, wherein the nRBCs are enriched in a flow-through microfluidic device.

12. The method of claim 1, 2 or 3, wherein the enumerating of nRBCs is performed by flow cytometry, fluorescence imaging, or radioactive imaging.

13. The method of claim 1, 2, or 3 further comprising performing fluorescence in situ hybridization on said nucleated red blood cells with chromosome-specific probes.

14. The method of claim 2, wherein when the number of nRBCs and/or aneuploid nRBCs exceeds a pre-determined value, said method further comprises determining the genetic characteristics of said pregnant woman's fetus.

15. The method of claim 3, wherein said serum markers is comprised of papA, free β HCG, unconjugated estriol (UE3), AFP, HCG, or inhibin.

16. The method of claim 2, wherein said aneuploidy is trisomy 21.

17. The method of claim 2, wherein said aneuploidy is trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY, or triploidy.

18. The method of claim 1 or 3, wherein said fetal abnormal condition is Klinefelter Syndrome, dup(17)(p11.2p1.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Prader-Willi syndrome, Kallmann syndrome, microphthalmia with linear skin defects, Adrenal hypoplasia, Glycerol kinase deficiency, Pelizaeus-Merzbacher disease, testis-determlining factor on Y, Azospermia (factor a), Azospermia (factor b), Azospermia (factor c), 1p36 deletion, or a combination thereof.

19. The method of claim 2, further comprising determining the origin of the cells enumerated in step (b).

20. The method of claim 2 wherein said sample is a peripheral blood sample.

21. The method of claim 2 wherein said sample is an amniotic sample.

22. A method for determining a condition in a fetus of a subject comprising:
enriching one or more nucleated red blood cells from a first sample from said subject;
performing a maternal serum marker screen on said first sample or a second sample from said subject;
optionally, performing a Nuchal Translucency (NT) sonographic test on said first sample, said second sample, or a third sample from said subject;
determining a condition of said fetus based on:
(1) the number of nucleated red blood cells isolated from said first sample;
(2) the results from said maternal serum marker screen; and
(3) optionally, the results from said Nuchal Translucency test.

23. The method of claim 22, wherein said condition is selected from the group consisting of trisomy 8, trisomy 9, trisomy 12, trisomy 13, trisomy 18, trisomy 21, XXX, XXY, XYY, XXXY, XXYY, XYYY, XXXXX, XXXXY, XXXYY, XXYYY, XYYYY, Klinefelter Syndrome, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p36 deletion, or a combination thereof.

24. The method of claim 22, wherein said first sample, second sample, or third sample is a peripheral blood sample.

25. The method of claim 22, wherein said material serum marker screen is AFP, MSAFP, Double Marker Screen, Double Screen, Triple Marker Screen, Triple Screen, Quad Screen, 1^{st} Trimester Screen, 2^{nd} Trimester Screen, Integrated Screen, Combined Screen, Contingency Screen, Repeated Measures Screen or Sequential Screen.

26. The method of claim 22, wherein said subject is under the age of 35.

27. The method of claim 22, wherein said sample is taken in the first trimester of pregnancy.

28. The method of claim 22, wherein said enriching is based on cell size and/or magnetic property.

29. The method of claim 28, wherein said enriching comprises using arrays of obstacles.

30. The method of claim 28, wherein said enriching comprises rendering nucleated red blood cells magnetic.

31. The method of claim 28, wherein said enriching comprises using arrays of obstacles and rendering nucleated red blood cells magnetic.

32. A method for determining the presence of a maternal abnormal condition comprising:
enumerating nucleated red blood cells in a blood sample from a pregnant woman; and
determining the presence of a maternal abnormal condition based on the number of nucleated red blood cells in the blood sample.

33. The claim of method 32, wherein the condition is Pre-eclampsia.


SEQUENCE LISTING (3 sequences)

SEQ ID NO: 1 - DNA, 19 bp, Artificial Sequence
  Description of Artificial Sequence Synthetic primer
tcgagtgcattccattccg

SEQ ID NO: 2 - DNA, 21 bp, Artificial Sequence
  Description of Artificial Sequence Synthetic primer
atggaatggcatcaaacggaa

SEQ ID NO: 3 - DNA, 15 bp, Artificial Sequence
  Description of Artificial Sequence Synthetic probe
tggctgtccattcca